(19) United States
(12) Patent Application Publication
(10) Pub. No.: US 2015/0004561 A1
(43) Pub. Date: Jan. 1, 2015

(54) METAL ARTIFACT REDUCTION FROM METALLIC DENTAL FILLINGS AND/OR CROWNS IN COMPUTED TOMOGRAPHY (CT) AND/OR X-RAY

(71) Applicant: KONINKLIJKE PHILIPS N.V., EINDHOVEN (NL)

(72) Inventor: Thomas Koehler, Norderstedt (DE)

(21) Appl. No.: 14/371,302

(22) PCT Filed: Jan. 22, 2013
(86) PCT No.: PCT/IB2013/050544

§ 371 Date: Jul. 9, 2014

Related U.S. Application Data
(60) Provisional application No. 61/590,354, filed on Jan. 25, 2012.

(51) Int. Cl. A61B 6/04; A61B 6/03; A61B 6/14

(52) U.S. Cl. CPC A61B 6/04; A61B 6/14; A61B 6/032
USPC 433/140; 433/215

(57) ABSTRACT

A method includes planning a head scan of a subject via a console (716) of an imaging system (700), wherein the head scan scans both jaws of the subject, including metallic dental fillings of teeth of the subject, scanning the subject 108, including the metallic dental fillings of the teeth of the subject, via the imaging system, wherein the subject has a dental metal artifact reduction device (802) installed in their mouth during the scan, and generating projection data indicative of the scanned region of the head of the subject, including the metallic dental fillings of the teeth of the subject.

The following generally relates to imaging, more particularly to reducing metal artifact in images from metallic dental fillings and/or crowns, finds particular application to computed tomography (CT), and is also amenable to other imaging modalities such as x-ray.

A CT scanner includes an x-ray tube mounted on a rotatable gantry that rotates around an examination region about a z-axis. A detector array subtends an angular arc opposite the examination region across from the x-ray tube. For a scan, the x-ray tube emits radiation that traverses the examination region and a portion of an object or subject therein. The detector array detects radiation that traverses the examination region and the portion of an object or subject, and generates projection data indicative thereof. A reconstructor reconstructs the projection data, generating volumetric image data indicative thereof. The volumetric image data can be processed to generate one or more images indicative of the scanned portion of the scanned object or subject.

With CT, as well as other imaging modalities, various artifacts can degrade the visual and diagnostic quality of the generated images. One source of such artifact is metallic material in the scanned portion of the scanned object or subject. Other sources of artifact include the imaging system, the image processing system, and/or other sources. Generally, metallic materials result in “streak” artifact, which occurs because filtered backprojection (FBP) algorithms assume that each detector measurement is equally accurate, when, in practice, x-ray beams that pass through or near metallic materials are highly attenuated relative to x-ray beams that do not pass through or near metallic materials.

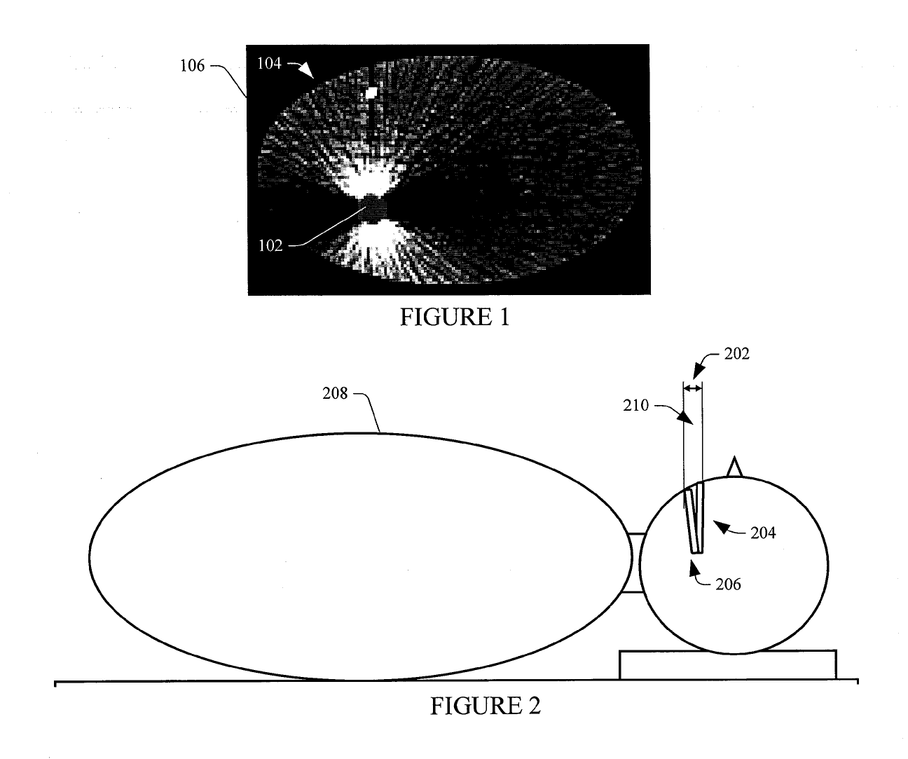

An example of such artifact caused by a metallic material is shown in FIG. 1. In FIG. 1, a metallic object 102 causes streaks 104 in a backprojected image 106, as well as dark or bright shading artifacts. Metal artifact reduction techniques exist. However, the results are often not suitable, in particular, if there are a lot of metallic objects within the field of view because large parts of the sinogram are affected. This situation is often present when the jaw with dental fillings is in the field of view. Typically, all dental fillings present in the upper or lower jaw are visible in the same slice, and if there are fillings or crowns on the incisors, the metal from both jaws may even be present in the same axial slices.

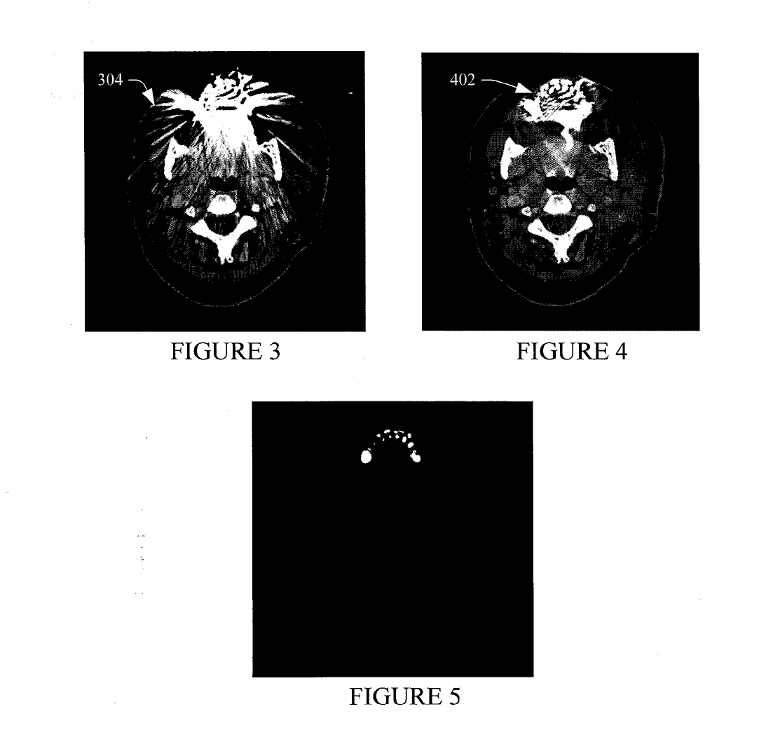

By way of example, FIG. 2 shows a z-extent 202 for an example head scan which covers both jaws 204 and 206 of a subject 208 where dental fillings of the subject 208 are scanned in the same acquisition interval 210, for purposes of explanation. FIG. 3 shows a reconstructed image with streak artifact (e.g. a streak 304 of a plurality of streaks) caused by the metallic fillings in teeth of the jaws 204 and 206. FIG. 4 shows the same image after applying a metal artifact reduction algorithm. Note that resulting image still includes artifact 402 and may not be suitable for diagnostic purposes. For reference, FIG. 5 shows an image created by segmenting the metallic filings from the image of FIG. 3. As can be seen, in this example, multiple metallic filings are in the same image.

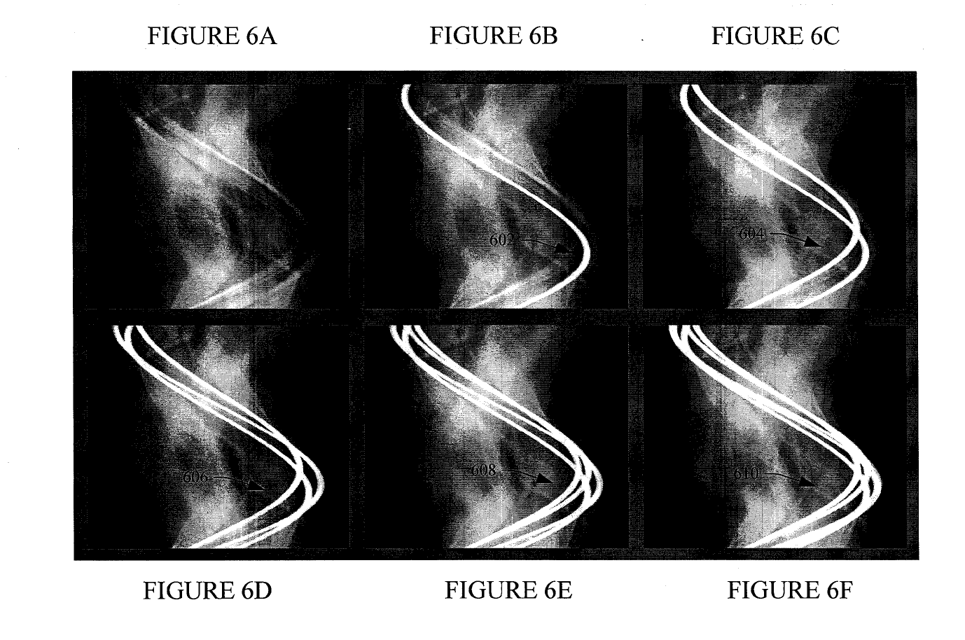

FIGS. 6A-6F respectfully show sinograms with increasing amount of metal shadow (e.g., 602, 604, 606, 608 and 610) caused by an increasing number of metallic filings in the acquired data. In this example, the number of metallic fillings increases from zero (0) to six (6) metallic fillings going from FIG. 6A to FIG. 6F. Unfortunately, the interpolation required across the shadow becomes increasingly more demanding with the increasing number of metallic fillings, and, as shown in FIG. 4, may not be able to be suitably removed from the resulting images.

Aspects described herein address the above-referenced problems and others.

In one aspect, a method includes planning a head scan of a subject via a console of an imaging system. The head scan scans both jaws of the subject, including metallic dental fillings of teeth of the subject. The method further includes scanning the subject, including the metallic dental fillings of the teeth of the subject, via the imaging system. During the scan, the subject has a dental metal artifact reduction device in their mouth. The method further includes generating projection data indicative of the scanned region of the head of the subject, including the metallic dental fillings of the teeth of the subject.

In another aspect, a dental metal artifact reduction device includes sides and a region therebetween. The sides position the jaws of a subject for a scan such that metallic dental fillings of the teeth of the subject are distributed along a z-extent in which at least some of the metallic dental fillings are imaged during different data acquisition intervals of the scan.

In another aspect, a method includes installing a dental metal artifact reduction device in the mouth of a subject to be scanned, wherein the installed dental metal artifact reduction device separates the jaws of the subject by a predetermined amount. The method further includes planning a head scan of the subject via a console of an imaging system, wherein the head scan scans both of the jaws of the subject, including the metallic dental fillings of teeth of the subject. The method further includes scanning the subject, including the metallic dental fillings of the teeth of the subject, via the imaging system, wherein the subject has a dental metal artifact reduction device installed in their mouth during the scan. The method further includes generating projection data indicative of the scanned region of the head of the subject, including the metallic dental fillings of the teeth of the subject.

The invention may take form in various components and arrangements of components, and in various steps and arrangements of steps. The drawings are only for purposes of illustrating the preferred embodiments and are not to be construed as limiting the invention.

FIG. 1 illustrates streak and shading artifacts in an image caused by a metallic structure in a scanned object.

FIG. 2 schematically illustrates an example head scan, showing the z-extent of an acquisition.

FIG. 3 shows a reconstructed image of a head scan over the z-extent of FIG. 2 with streak artifact caused by metallic dental fillings.

FIG. 4 shows the results of processing the reconstructed image of FIG. 3 with a metal artifact reduction algorithm.

FIG. 5 shows a segmentation of the metallic fillings of the reconstructed image of FIG. 5.

FIGS. 6A-6F shows sinograms with shadows for acquisitions covering zero (0) to six (6) metallic fillings.

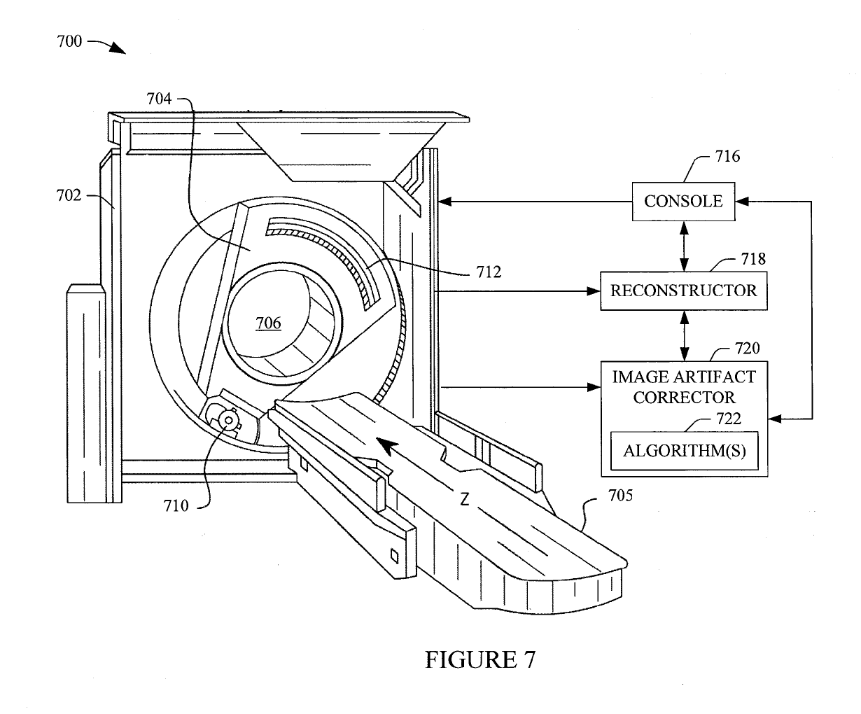

FIG. 7 schematically illustrates an imaging system in connection with a dental metal artifact device.

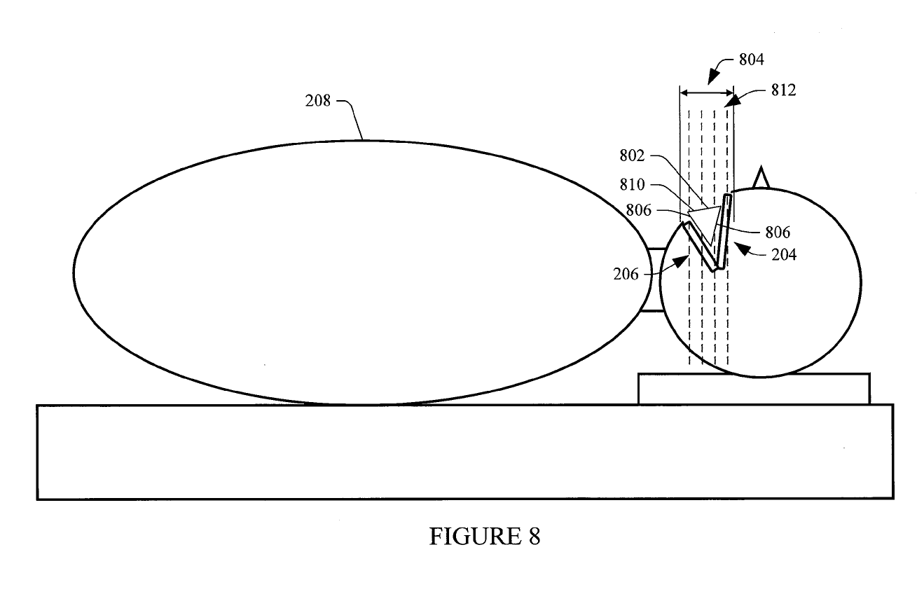

FIG. 8 schematically illustrates an example head scan, showing the z-extent of an acquisition using the dental metal artifact reduction device.

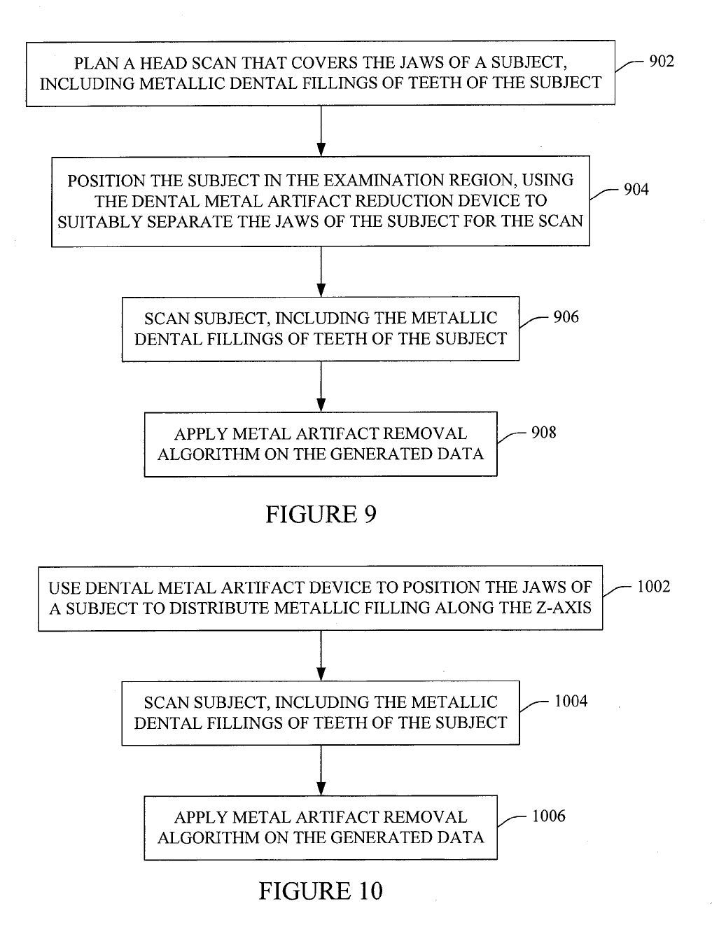

FIG. 9 illustrates an example method using the dental metal artifact reduction device described herein.

FIG. 10 illustrates another example method using the dental metal artifact reduction device described herein.

FIG. 7 illustrates an example imaging system 700, such as a computed tomography (CT) scanner. The imaging system 700 includes a generally stationary gantry 702 and a rotating gantry 704. The rotating gantry 704 is rotatably supported by the stationary gantry 702 and rotates around an examination region 706 about a z-axis. A subject support 705, such as a couch, supports an object or subject in the examination region 706. In one non-limiting instance, the subject is scanned using a head or other imaging protocol in which metallic dental fillings of the subject are in the examination region during scanning

A radiation source 710, such as an x-ray tube, is rotatably supported by the rotating gantry 704. The radiation source 710 rotates with the rotating gantry 704 and emits radiation that traverses the examination region 706 and a sub-portion of the object or subject therein. A one or two dimensional radiation sensitive detector array 712 subtends an angular arc opposite the radiation source 710 across the examination region 706. The detector array 712 detects radiation traversing the examination region 706 and the sub-portion of the object or subject, and generates projection data indicative thereof.

A general-purpose computing system or computer serves as an operator console 716. The console 716 includes a human readable output device such as a monitor and an input device such as a keyboard, mouse, etc. Software resident on the console 716 allows the operator to interact with and/or operate the scanner 700 via a graphical user interface (GUI) or otherwise. Such interaction may include selecting an imaging protocol such as a head protocol, selecting an image artifact removal algorithm, etc. A reconstructor 718 reconstructs the projection data and generates volumetric image data indicative thereof.

An artifact corrector 720 employs at least one metal artifact correction algorithm 722 to correct for streak artifact caused by a metallic object (e.g., a metallic dental filling and/or crown) in the examination region 706 during scanning In one instance, the artifact corrector 720 employs an image domain algorithm which corrects for the streak artifact in the image domain. In another instance, the artifact corrector 720 employs a projection domain algorithm which corrects for the streak artifact in the projection domain. In yet another instance, the artifact corrector 720 employs an algorithm which goes back and forth between the projection and image domains to correct for the streak artifact.

An example of a suitable metal artifact reduction approach is described in U.S. Pat. No. 7,340,027 B2, filed Jun. 16, 2004, and entitled “Metal Artifact Correction in Computed Tomography,” the entirety of which is incorporated herein by reference. Another example of a suitable metal artifact reduction approach is described in PCT publication WO 2008084352 A1, filed Dec. 19, 2007, and entitled “Apparatus, Method and Computer Program for Producing a Corrected Image of a Region of Interest from Acquired Projection Data,” the entirety of which is incorporated herein by reference. Other approaches are also contemplated herein.

As briefly discussed above, in one non-limiting instance, the subject is scanned using a head or other imaging protocol in which metallic dental fillings (and/or crowns) of the subject are in the examination region during scanning Also discussed above, metallic dental fillings (and/or crowns), when located in the examination region 706 during a scan, may cause streak artifact (e.g., as shown in FIG. 3) that cannot be corrected very well by known metal artifact reduction techniques (e.g., as demonstrated in FIG. 4), especially where multiple filling (and/or crowns) are concurrently present in the examination region 706 such that they are present in the same sinogram and image of the reconstructed image data.

FIG. 8 shows an embodiment in which a dental metal artifact reduction device 802 is utilized in connection with the subject 208 to position the jaws 204 and 206 of the subject with respect to each other so that metallic dental fillings (and/or crowns) of the subject 208, for a z-extent 804, are distributed over multiple data acquisition intervals 812 (and hence images) relative to FIG. 2, where the same data acquisition interval 210 images all of the fillings (and/or crowns). In this manner, some of the metallic dental fillings (and/or crowns) are imaged during different data acquisition intervals, and the sinogram corresponding to any of the multiple data acquisition intervals will have less shadowing (e.g., compare FIG. 6A and FIG. 6F), such that the interpolation required across the shadow is not too demanding for conventional metal artifact correction algorithms to suitable remove metal artifacts.

In this example, the dental metal artifact reduction device 802 is wedge-shaped (or shaped to easily and comfortably slide into an open mouth of the subject 108). The device 802 includes a first side 806 and a second side 808 separated by a region 810 which generally increases in width, from a point at which the two sides converge, along a transverse direction of the device 802, which is perpendicular to the z-direction of the device 802, forming the wedge. In the illustrated embodiment, the wedge has linear sides which are equal in length. However, in other embodiment, at least one of the sides can be curved or otherwise shaped, and the sides do not have to be increasingly separated or have the same length. Where the device 802 is specific to the subject, one or both of the sides may include recesses corresponding to the teeth. This may facilitate placement and/or maintaining placement in the mouth.

The geometry of the illustrated wedge is such that an installed dental metal artifact reduction device 802 fills the void or space between the palate and the tongue, which mitigates tongue motion and thus motion artifact caused by a moving tongue during the scan. That is, the dental artifact reduction device has a geometry that inhibits movement of the tongue of the subject when in the mouth of the subject during the scan. However, it is to be understood that the illustrated geometry (i.e., shape and size) of the dental metal artifact reduction device 802 is provided for explanatory purposes and is not limiting. As such, in other embodiments, the dental metal artifact reduction device 802 may have other geometries. Although the dental metal artifact reduction device 802 is shown as a single element, it is to be appreciated that is may include multiple elements, which are assembled to form the device 802, which are individually placed in the mouth of the subject 208, and/or are otherwise employed in connection with each other.

Moreover, the dental metal artifact reduction device 802 includes a material that is generally radio lucent, e.g. made of Rohacell, such that x-rays traversing the dental metal artifact reduction device 802 are substantially unattenuated. In one instance, this results in images that, visually, with respect to intensity, are similar to images acquired without the dental metal artifact reduction device 802. Less radiolucent material can alternatively be used. It is to be appreciated that a single generic dental metal artifact reduction device 802 can be used with all subjects, multiple different generic dental metal artifact reduction devices 802 can correspond to different types or categories of subject (e.g., adult, child, and infant), a single dental metal artifact reduction device 802 can be specific to a particular subject, etc. In one instance, the device 802 includes multiple members which are removably affixed to each other, and one or more of the members may be removed or added to the device 802 to change the geometry of the device 802.

In one instance, the operator of the imaging system 700, when planning a head scan for the subject 208, enters, via the console 716, information about the dental metal artifact reduction device 802. Such information can be used to automatically populate and/or recommend one or more parameters of the plan. For instance, the information my include a serial number or unique identifier which is used to obtain a length of the dental metal artifact reduction device 802 in the z-direction, and this information can be used to set the z-extent, a number of slices to reconstruct, a slice width, a metal artifact reduction algorithm 722, etc.

In another instance, where the dental metal artifact reduction device 802 includes a wireless transmitter (e.g., an RFID tag) and the console 716 include a corresponding wireless receiver (e.g., an RFID receiver), the information about the dental metal artifact reduction device 802 and/or the corresponding subject can be transmitted from the dental metal artifact reduction device 802 to the console 716. Again, this information can be used to automatically populate and/or recommend one or more parameters of the plan.

In another instance, after planning the scan, the system 700 visually displays a serial number, name and/or unique identifier of a dental metal artifact reduction device 802 which the system 700 recommends to use for the scan. For example, the system 700 may recommend a particular dental metal artifact reduction device 802 based on the length of the dental metal artifact reduction device 802 in the z-direction in the plan.

FIG. 9 illustrates an example method.

It is to be appreciated that the ordering of the acts in the methods described herein is not limiting. As such, other orderings are contemplated herein. In addition, one or more acts may be omitted and/or one or more additional acts may be included.

At 902, a head scan, which covers the jaws 204 and 206 of the subject 208, including the metallic dental fillings of the subject 108, is planned for the subject 208 via the console 716.

At 904, the subject 208 is positioned in the examination region 706 via the subject support 705 based on planned scan start and end points.

In this example, the dental metal artifact reduction device 802 is in the mouth of the subject 208, positioning the jaws 204 and 206 such that the metallic dental fillings of the subject 208 are distributed along a z-extent that is longer than a z-extent if the jaws 204 and 206 were not separated as such, distributing the metallic dental fillings over more data acquisition internals and hence reconstructed images.

At 906, the subject 108 is scanned via the imaging system 700 based on the planned head scan, including scanning the jaws 204 and 206 and the metallic dental fillings of the teeth therein.

At 908, the projection and/or image data from the scan is processed via the image artifact corrector 720 to remove metal artifact using one of the metal reduction artifact algorithms 722 as discussed herein.

FIG. 10 illustrates another example method.

It is to be appreciated that the ordering of the acts in the methods described herein is not limiting. As such, other orderings are contemplated herein. In addition, one or more acts may be omitted and/or one or more additional acts may be included.

At 1002, the dental metal artifact reduction device 802 is placed in the mouth of the subject 108, positioning the jaws 204 and 206 such that the metallic dental fillings of the subject 108 are distributed along a predetermined z-extent that is longer than a z-extent if the jaws 204 and 206 were not separated, distributing the metallic dental fillings over more data acquisition internals and hence reconstructed images.

At 1004, the subject 108 is scanned via the imaging system 700, including the jaws 204 and 206 and the metallic dental fillings of the teeth therein.

At 1006, the projection and/or image data from the scan is processed via the image artifact corrector 720 to remove metal artifact using one of the metal reduction artifact algorithms 722 as discussed herein.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A method, comprising
planning a head scan of a subject via a console of an imaging system, wherein the head scan scans both jaws of the subject, including metallic dental fillings of teeth of the subject;
scanning the subject, including the metallic dental fillings of the teeth of the subject, via the imaging system, wherein the subject has a dental metal artifact reduction device installed in their mouth during the scan; and
generating projection data indicative of the scanned region of the head of the subject, including the metallic dental fillings of the teeth of the subject

2. The method of claim 1, wherein the dental metal artifact reduction device positions the jaws for the scan such that the metallic dental fillings are distributed along a z-extent in which at least some of the metallic dental fillings are imaged during different data acquisition intervals.

3. The method of claim 1, wherein the dental metal artifact reduction device positions the jaws for the scan such that the metallic dental fillings are distributed along a first z-extent which is longer than a second z-extent used to scan the subject without the dental metal artifact reduction device.

4. The method of claim 1, further comprising:
applying a projection domain metal artifact reduction algorithm to the projection data, thereby reducing metal artifact caused by the metallic dental fillings.

5. The method of claim 1, further Comprising:
reconstructing the projection data, producing image data; and
applying an image domain metal artifact reduction algorithm to the image data, thereby reducing metal artifact caused by the metallic dental fillings.

6. The method of claim 1, wherein the dental metal artifact reduction device includes a material which is substantially radiolucent.

7. The method of claim 1, wherein the dental metal artifact reduction device has a geometry that inhibits movement of the tongue of the subject when the dental metal artifact reduction device is installed in the mouth of the subject.

8. The method of claim 1, wherein a single generic dental metal artifact reduction device is used for all subjects.

9. The method of claim 1, wherein the dental metal artifact reduction device is specific to an individual subject.

10. The method of claim 1, wherein the dental metal artifact reduction device is one of a plurality of metal artifact reduction devices, each corresponding to a different class of subjects.

11. The method of claim 1, wherein the dental metal artifact reduction device is wedge shaped.

12. A dental metal artifact reduction device, comprising:
sides and a region therebetween, wherein the sides position the jaws of a subject for a scan such that metallic dental fillings of the teeth of the subject are distributed along a z-extent in which at least some of the metallic dental fillings are imaged during different data acquisition intervals of the scan.

13. The dental metal artifact reduction device of claim 12., wherein the dental metal artifact reduction device further positions jaws for the scan such that the metallic dental fillings are distributed along a first z-extent which is longer than a second z-extent used to scan the subject without the dental metal artifact redaction device.

14. The dental metal artifact reduction device of claim 12, wherein the dental metal artifact reduction device includes a material which is substantially radiolucent.

15. The dental metal artifact reduction device of claim 12, wherein the dental metal artifact reduction device has a geometry that inhibits movement of the tongue of the subject when the dental metal artifact reduction device is installed in the mouth of the subject.

16. The dental metal artifact reduction device of claim 12, wherein the dental metal artifact reduction device includes a single generic dental metal artifact reduction device that is used for all subjects,

17. The dental metal artifact reduction device of claim 12, wherein the dental metal artifact reduction device is specific to a single individual subject.

18. The dental metal artifact reduction device of claim 12, wherein the dental metal artifact reduction device is one of a plurality of metal artifact reduction devices, each corresponding to a different class of subjects.

19. A method, comprising:
installing a dental metal artifact reduction device in the mouth of a subject to be scanned, wherein the installed dental metal artifact reduction device separates the jaws of the subject by a predetermined amount;
planning a head scan of the subject via a console of an imaging system, wherein the head scan scans both of the jaws of the subject, including the metallic dental fillings of teeth of the subject;
scanning the subject, including the metallic dental fillings of the teeth of the subject, via the imaging system, wherein the subject has a dental metal artifact reduction device installed in their mouth during the scam and
generating projection data indicative of the scanned region of the head of the subject, including the metallic dental fillings of the teeth of the subject.

20. The method of claim 19, wherein the dental metal artifact reduction device separates the jaws for the scan such that the metallic dental fillings are distributed along the z-extent such that at least some of the metallic dental fillings are imaged during different data acquisition intervals.
